(19) United States
(12) Patent Application Publication
(10) Pub. No.: US 2009/0131391 A1
(43) Pub. Date: May 21, 2009

(54) INHIBITOR FOR DIFFERENTIATION OF HEMATOPOIETIC PRECURSOR CELLS

(75) Inventors: Rakhimdzhan A. ROZIEV, Obninsk (RU); Anna Y. GONCHAROVA, Obninsk (RU); Vladimir K. PODGORODNICHENKO, Obninsk (RU); Alexandr S. SAENKO, Obninsk (RU); Irina A. ZAMULAEVA, Obninsk (RU); Olga V. SEMINA, Obninsk (RU); Yurij A. Semin, Obninsk (RU); Tamara N. SEMENETS, Obninsk (RU); Nina V. ORLOVA, Obninsk (RU); Svetlana G. SMIRNOVA, Obninsk (RU); Anatoliy F. TSYB, Obninsk (RU)

Correspondence Address: HOUSTON ELISEEVA, 4 MILITIA DRIVE, SUITE 4, LEXINGTON, MA, 02421, US

(73) Assignee: OOO "MBF", Obninsk (RU)

(21) Appl. No.: 12/210,416

(22) Filed: Sep. 15, 2008

Related U.S. Application Data
(63) Continuation of application No. PCT/RU2007/000118, filed on Mar. 13, 2007.

(30) Foreign Application Priority Data
Mar. 16, 2006 (RU) 2006108074

(51) Int. Cl. A61K 31/33

(52) U.S. Cl. 514/183

(57) ABSTRACT

The invention relates to medicine and can be used for treating persons with diseases associated with a differentiation of hematopoietic precursor cells. The inventive inhibitor for differentiation of hematopoietic precursor cells is embodied in the form of an organic compound of selen-9-phenyl-symmetrical-octahydro-selenoxanten. Said agent makes it possible to more efficiently protect normal tissues.

### RELATED APPLICATIONS

This application is a Continuation of PCT application serial number PCT/RU2007/000118, filed on Mar. 13, 2007 which in turn claims priority to Russian Patent Application No. RU 2006108074 filed on Mar. 16, 2006, both of which are incorporated herein by reference in their entirety.

### FIELD OF THE INVENTION

This invention pertains to the field of Medicine/medical science; it can be used for treatment and prevention of conditions associated with hematopoietic precursor (progenitor) cell differentiation.

### BACKGROUND OF THE INVENTION

Hematopoiesis is a complex multi-stage process of cell division and differentiation resulting in mature and functional blood cells. Blood is a highly dynamic system, a unique and constantly renewed tissue, capable of rapid and precise response to the organism's changing requirements.

There are 6 major classes of blood cells, three of which include hematopoietic progenitor cells. The earliest hematopoietic cells that give rise to all blood cell types constitute the stem cell class. Hematopoietic microenvironment is required for stem cell differentiation and proliferation; the microenvironment consists of stromal cells (macrophages, fibroblasts, endothelial, adipose and reticular cells), micro-vessels and the extracellular matrix (fibronectin, hemonectin, laminin, collagen and mucopolysaccharides like heparan sulfate).

Next cell class includes the closest stem cell derivatives—pluripotent and committed progenitor cells with a more limited differentiation potential then that of a stem cell. These cells can form colonies in a variety of culture media and are, therefore, termed colony-forming cells (CFC's). Unipotent progenitor cells constitute the final class and can only differentiate within a given cell lineage.

Known methods of protecting the hematopoietic organs against cytotoxic effects of chemotherapy and radiation treatment make use of a variety of substances most of which are antioxidants.

Antioxidants improve cells' resistance to toxic agent via two distinct mechanisms. The major mechanism relies on ability of antioxidants to sequester free radicals, which accumulate inside a cell, primarily during irradiation. This mechanism is only effective if the antioxidant is present in a cell at the time of free radical formation, i.e. during exposure to ionizing radiation or cytostatic agents. The effectiveness of this mechanism is generally directly proportional to the intracellular antioxidant concentration. These protective properties are effective against both low and high doses of radiation. Main drawback of this protective mechanism is that when introduced into the organism, antioxidants confer protection upon malignant cells as well as the healthy tissue. This limits use of antioxidants as protective agents for hematopoietic and other cells during anti-tumor therapy as their protective properties may negate as much as two thirds of the effectiveness of radiation.

The second mechanism of radiation resistance is described in Patent [RU 2234918/A61K 31/375, 2004/] and is focused in anti-oxidative properties. This mechanism relies on alteration of the cell's biological properties upon introduction of antioxidants and development of so-called adaptive response. The adaptive response is only initiated by introduction of specific antioxidant doses and generally manifests the exogenous antioxidant has been eliminated from cells. This mechanism's merit is in its manifestation duration. The effect is observed as early as 4 hours after the introduction of the agent and persists to over 7 days. Main drawback is that the method is effective with low dose radiation; the cells' radiation resistance increases no more than 1.3-1.5 fold.

Presently the scientific efforts are focused of studies of progenitor cells in animals that underwent various oncological and radiation treatments.

It is thought that stem cells and other progenitor cells are important for the disease progression, particularly carcinogenesis.

Hematopoietic growth factors regulate proliferation and differentiation of progenitor cells as well as function of the mature blood cells. These factors, including erythropoietin and growth factor likver, are presently used in medical practice and are the only stimulating factors of progenitor cell growth and differentiation used in cases of various immune deficiencies.

The inhibitors of the above functions of the hematopoietic progenitor cells are equally important. It was shown that adjustment of stem cell differentiation and proliferation in vitro by an inhibitor can improve conditions for auto-transplantation of hematopoietic cells in cases of autoimmune pathology.

The closest analog of the above method is a following method of differential protection of normal mammalian cells against chemo- or radiation therapy: a polypeptide stem cell proliferation inhibitor composition is chosen from an array of molecules including alpha-, beta-, gamma-, delta-, epsilon- and zeta-globin hemoglobin chains, and a suitable pharmaceutical delivery system (RU 2186579 C1, 2002 Aug. 10). This method of regulation influences proliferation of stem cells belonging to a particular type of hematopoietic progenitor cells.

Presently we do not know of any treatments for conditions accompanied by cell differentiation, which would produce a temporary inhibition of the differentiation and thus, have a protective effect on normal tissues.

### SUMMARY OF THE INVENTION

The purpose of this invention is to create a method for temporary inhibition of hematopoietic progenitor cell differentiation.

We propose to inhibit progenitor cell differentiation by introduction of a selenium compound, 9-phenyl-symmetric-octahydroselenoxanthene (molecular structure below)

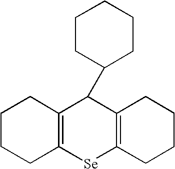

Specifically, utilization of 9-phenyl-symmetric-octahydroselenoxanthene as a differentiation inhibitor allows protection of progenitor cells of the bone marrow against radiation doses below 1.5 Gy and against effects of cytostatic agents.

In cases where progenitor cell differentiation is associated with a tumor, which is being treated with radiation or chemotherapy, 9-phenyl-symmetric-octahydroselenoxanthene is introduced once 5-9 (preferably 7) days prior to treatment. The agent may be administered orally or by injection at 0.1 to 5 mg/kg of [patient's] weight.

Differentiating progenitor cells are at the differentiation stage at the time of application of therapy (radiation or cytostatic) and thus are more resistant to the effects of cytostatic agents.

The proposed agent's biological activity is mainly due to its molecular structure rather than to presence of selenium. The following support this statement:

Xanthene-like compounds (which include the proposed agent) have a wide spectrum of biological properties. Their activity is directly related to their molecular structure. Certain thioxanthenes and thioxanthones are highly effective in treatment of schistomatosis; however, if methyl group is substituted for a methoxyl group or a chlorine atom all biological activity is lost. (Harchenko V G, Kirupina T I, Blinohvatov A F. Thioxanthenes, Hydroxanthenes and Their Derivatives Saransk University Press 1979, pp. 68-71)

Further, various xanthene-like compounds exhibit a range of effects: antimicrobials, depressants and anti-depressants, anti-histamines and fever-reducing agents, anti-tumor agents. Thus, the chemical structure of these compounds defines their function.

All of the compounds mentioned above do not contain selenium. Xanthene compounds were found to be an excellent precursor for biologically active compounds (agents).

The proposed agent (compound) is a xanthene-like compound, which, however, contains selenium. It was previously shown that this compound activates certain physiological systems, however, there are no previous mention of the proposed properties described here. We cannot exclude a possibility that the observed effects are at least partially due to presence of selenium; however, it is evident that in this case the whole is greater than the sum of its parts, and, therefore, we are observing novel properties of the compound.

9-phenyl-symmetric-octahydroselenoxanthene can be produced by a previously described method (RU 2221793, published Jan. 20, 2004).

To prove the invention's applicability experiments were conducted in mice to determine the sensitivity of the bone marrow cells to the impacts of various agents after introduction of the proposed agent in recommended dose.

### DETAILED DESCRIPTION OF THE PREFERRED EMBODIMENTS

### Example 1

Assay of effect of various doses of ionizing radiation on hematopoiesis. 950 female mice of two lines—(CBA×C 57 B 1/6) F1 and (DBA×C 57B1/6) F1—were used in this study. Radiation was administered using a radiation therapy machine “Luch-1” utilizing gamma-rays ⁶⁰Co at 0.9 Gy/min. 9-phenyl-symmetric-octahydroselenoxanthene was administered orally in doses 1.0, 5.0, 10 and 20 mg/kg in 0.2 ml of oil. Agent was administered 1, 3 or 7 days prior to radiation treatment of 1.5 Gy; bone marrow was then extracted from donor mice, and cell suspension was injected into lethally irradiated (8 Gy) recipient mice of the same line. Colony-forming activity was assayed by number of exogenous spleen colonies when mice were sacrificed on day 9.

TABLE 1
Effects of 5 mg/kg of 9-phenyl-symmetric-octahydroselenoxanthene
administered at various time points prior to radiation treatment
with 1.5 Gy on development of adaptive response (as measured
by number of colony-forming cells in the spleen, CFC-S)

| | | | Number of | |
|---|---|---|---|---|
| | Time of agent | | colonies formed | |
| | administration, | | per 10⁵ of bone | |
| Irradiation | days prior to | Number of | marrow cells, | |
| 1.5 Gy | treatment | mice | M ± m | p value |
| − | — | 30 | 10.5 ± 0.6 | |
| − | 1 | 30 | 12.5 ± 0.6 | |
| − | 3 | 30 | 10.7 ± 0.4 | |
| − | 7 | 30 | 11.1 ± 0.7 | |
| + | — | 30 | 3.6 ± 0.3** | |
| + | 1 | 30 | 6.4 ± 0.5* | p < 0.001 |
| + | 3 | 30 | 9.1 ± 0.5* | p < 0.0001 |
| + | 7 | 30 | 12.7 ± 0.7* | p < 0.0001 |

**validity calculated relative to given group

Results presented in Table 1 (averaged over three independent experiments) show that administration of 5.0 mg/kg of 9-phenyl-symmetric-octahydroselenoxanthene 1, 3 or 7 days prior to radiation treatment at permissive dose of 1.5 Gy produces a CFC-S adaptive response to the ionizing radiation—cell survival is significantly higher than that of the irradiated control. The effect persists for 7 days. The time of the administration was noted to be important: minimal effect (a 2-fold increase in CFC-S compared to the irradiated control) is observed when the agent is administered 1 day prior to radiation treatment; maximal effect (3-fold decrease of the effects of radiation) is achieved when the agent is administered 7 days prior. The administration of 5.0 mg/kg of the agent alone at various time points prior to bone marrow extraction has no effect on CFC-S number in donor mice.

To assay ability of 9-phenyl-symmetric-octahydroselenoxanthene to restore stem cell population at higher degree of damage we assayed the agent's influence on development of adaptive response to different radiation doses.

300 female mice of line (CBA×C57B1/6) F1 were used in the experiment. Radiation was administered using a radiation therapy machine “Luch-1” utilizing gamma-rays ⁶⁰Co at 0.9 Gy/min. 9-phenyl-symmetric-octahydroselenoxanthene was administered orally in doses 1.0, 5.0, 10 and 20 mg/kg in 0.2 ml of oil 7 days prior to radiation treatment at following doses: 1.5, 2.0, 3.0 and 5.0 Gy. Bone marrow was extracted 10-15 minutes after the treatment and the cell suspension was injected into lethally irradiated (8 Gy) recipient mice. Colony-forming activity was assayed by number of exogenous spleen colonies when mice were sacrificed on day 9.

Results presented in Table 2 (averaged over three independent experiments) show that 7 days after administration of 5.0 mg/kg of 9-phenyl-symmetric-octahydroselenoxanthene a statistically significant CFC-S adaptive response to radiation of 1.5, 2.0, 3.0 and 5.0 Gy is developed. Cell survival at all radiation doses (excluding 1.5 Gy) is 2-fold of that of the irradiated control. At 1.5 Gy cell survival is not significantly different from that of the untreated control.

TABLE 2
Effects of administration of 9-phenyl-symmetric-octahydroselenoxanthene
7 days prior to radiation treatment with a range of doses on development
of adaptive response (as measured by number of colony-forming
cells in the spleen, CFC-S)

| | | | Number of | |
|---|---|---|---|---|
| | | | colonies formed | |
| Radiation | Proposed | | per 10⁵ of bone | |
| dose | agent | Number of | marrow cells, | |
| Gy | 5 mg/kg | mice | M ± m | P value |
| — | − | 30 | 10.3 ± 0.7 | |
| 1.5 | − | 30 | 3.5 ± 0.2 | P < 0.00001 |
| 1.5 | + | 30 | 9.2 ± 0.2 | |
| 2.0 | − | 30 | 2.7 ± 0.1 | P = 0.016 |
| 2.0 | + | 30 | 3.6 ± 0.3 | |
| 3.0 | − | 30 | 1.4 ± 0.1 | P = 0.009 |
| 3.0 | + | 30 | 2.1 ± 0.1 | |
| 5.0 | − | 30 | 0.3 ± 0.05 | P = 0.01 |
| 5.0 | + | 30 | 0.6 ± 0.06 | |

### Example 2

### Modifying Effects of the Proposed Inhibitor in Animals Exposed to Non-Ionizing Radiation

Protective properties of 9-phenyl-symmetric-octahydroselenoxanthene against non-ionizing radiation were examined by studying formation of micronuclei in polychromatophil mouse erythrocytes (line C57 B1). Micronuclei of the interphase nucleus are acentric chromosome fragments. Animals were exposed to electromagnetic radiation in millimeter wavelength range, frequency 39.5 GHz, and wavelength 7.5 mm for 1 hour. Flux density was approximately 3 microW/cm², which is 300 fold less than levels posing danger of overheating due to field energy absorption to a biological object.

5 mg/kg of 9-phenyl-symmetric-octahydroselenoxanthene was administered to group 1 (n=5) orally in 0.2 ml of oil 7 days prior to irradiation. Group 2 (n=5) received 0.2 ml of oil only and untreated mice were used as controls. Mice were sacrificed 1 day after irradiation and number of micronuclei was assayed in peripheral polychromatophil erythrocytes (150,000 cells per sample). Results are presented in Table 3.

TABLE 3
Frequency of micronuclei-containing peripheral polychromatophil
erythrocytes in mice treated or untreated with 9-phenyl-symmetric-
octahydroselenoxanthene and exposed to electromagnetic irradiation.

| | Frequency of micronuclei-containing |
|---|---|
| Treatment | polychromatophil erythrocytes × 10³ |
| Untreated | 2.8 ± 0.48 |
| Electromagnetic irradiation | 12.2 ± 1.28 |
| Irradiation + inhibitor | 6.0 ± 2.10 |

Thus, the proposed inhibitor 9-phenyl-symmetric-octahydroselenoxanthene confers protection not only against ionizing radiation but also against other radiation types.

### Example 3

The effects of the proposed agent (9-phenyl-symmetric-octahydroselenoxanthene) on mouse hematopoiesis were assayed by flow cytometry. Relative number of CD34+ cells and the density of the antigen were assayed in peripheral blood cells (80,000 cells per sample) and bone marrow cells (40,000 cells per sample). As reported earlier, administration of the proposed agent has no effect on relative number of less differentiated stem cells (CD34+). However, by day 7 after the agent administration the density of CD34 antigen on the cell surface increases (p<0.05) which suggests redistribution of the levels of differentiation in the cell population due to decrease in the least differentiated portion. The results seen in blood cells are also similar: according to three experiments, the portion of CD34+ cells on the blood does not change after 9-phenyl-symmetric-octahydroselenoxanthene administration; however, the number of more differentiated progenitors decreases after 7 days.

Total numbers of CD34+ cells are presented in Table 4.

TABLE 4
Number of CD34+ cells.

| | |
|---|---|
| | Total CD34+ |
| Total CD34+ | cells per 1 ml |
| cells in bone | of peripheral |
| marrow, 10⁷ | blood, 10³ |

| Treatment | Mean | ±SD | Mean | ±SD |
|---|---|---|---|---|
| Control | 3.6 | 2.0 | 10.5 | 5.6 |
| Agent administered 1 day | 4.0 | 3.0 | 6.8 | 4.2 |
| prior to analysis | | | | |
| Agent administered 7 days | 4.0 | 3.0 | 8.0 | 4.0 |
| prior to analysis | | | | |
| Gamma-irradiation, 1.5 Gy | 1.4 | 1.0 | 7.8 | 5.6 |
| Agent administered 1 day | 2.1 | 0.6 | | |
| prior to analysis + | | | | |
| irradiation, 1.5 Gy | | | | |
| Agent administered 7 days | 1.0 | 0.6 | 3.8 | 1.8 |
| prior to analysis + | | | | |
| irradiation, 1.5 Gy | | | | |

Analysis of qualitative and quantitave data on cellular composition of peripheral blood and bone marrow in animals after irradiation suggests that 9-phenyl-symmetric-octahydroselenoxanthene blocks differentiation of progenitor cells of the granulocyte/macrophage lineage. 7 days after the administration of the agent at the time of irradiation less differentiated progenitors—those least susceptible to the harmful effects of radiation—accumulate in the bone marrow. It is possible that irradiation and subsequent evacuation of hematopoietic organs reverses this differentiation block and, thus, leads to increase in leukocytes, granulocytes and monocytes in the peripheral blood on day 2 following irradiation.

### Example 4

The following experiments evaluate the effects of 9-phenyl-symmetric-octahydroselenoxanthene on tumor cells concurrent with standard Platidiam (cisplatin) treatment.

Results of the experiments are summarized in Table 5.

TABLE 5
Effects of inhibitor 9-phenyl-symmetric-octahydroselenoxanthene
on therapeutic action of Platidiam (cisplatin), tumor growth and
metastasis in C57B1/6 mice with LLC (Lewis lung carcinoma).

| | | | Avg number |
|---|---|---|---|
| | Tumor | Tumor | of lung |
| | mass, g | volume, sm³ | metastases |
| Experimental group | M ± m | M ± m | M ± m |
| Control | 6.4 ± 0.5 | 6.2 ± 0.7 | 8.8 ± 0.6 |
| Platidiam (cisplatin) | 4.5 ± 0.5 | 4.6 ± 0.6 | 6.2 ± 0.7 |
| Inhibitor + Platidiam | 5.3 ± 0.3 | 5.1 ± 0.4 | 5.4 ± 0.5 |
| (cisplatin) | | | |

Bold highlights significant difference (p < 0.05) with control group.

As shown above, Platidiam (cisplatin) has a slight but consistent anti-tumor effect by two of the parameters—tumor mass and lung metastases. Administration of 9-phenyl-symmetric-octahydroselenoxanthene 30 minutes prior to chemotherapy did not exhibit any significant effect on tumor growth and number of lung metastases. Thus, in given conditions, 9-phenyl-symmetric-octahydroselenoxanthene has no effect on growth and metastasis of a tumor treated with standard Platidiam (cisplatin) therapy. Since given concentration of the inhibitor is capable of reducing toxic effects of Platidiam (cisplatin) in normal tissues, it may justify using 9-phenyl-symmetric-octahydroselenoxanthene a selective protective agent for normal tissues during chemotherapy treatment of malignant tumors.

As was shown by previous experiments, the inhibitor does not affect tumor growth, even though the values for the growth index are slightly smaller in the “Inhibitor” group they are not statistically significant. The same is true for the combination of 9-phenyl-symmetric-octahydroselenoxanthene and irradiation: growth indexes of 9-phenyl-symmetric-octahydroselenoxanthene alone and 9-phenyl-symmetric-octahydroselenoxanthene+irradiation are not significantly different.

The number of lung metastases was assayed on day 21 in surviving animals. Data in Table 5 shows that 9-phenyl-symmetric-octahydroselenoxanthene does not influence metastatic processes significantly in neither non-irradiated nor in irradiated (tumor exhibiting) mice.

The second series of experiments produced similar results.

As in the previous series, tumor cells were injected subcutaneously into right hind leg at 2.0×10⁶ cells/mouse (total of 40 mice).

Concurrent use of the proposed agent 9-phenyl-symmetric-octahydroselenoxanthene and localized irradiation does not protect the tumor cells from effects of radiation and does not significantly increase metastasis.

TABLE 6
Growth index values of Lewis lung carcinoma (LLC) after administration of the inhibitor
(9-phenyl-symmetric-octahydroselenoxanthene) and localized irradiation.

| | Growth index | | | | | |
|---|---|---|---|---|---|---|
| Experimental group | | 1 day | 4 days | 6 days | 8 days | 11 days |
| Control | 1 | 1.84 ± 0.05 | 3.8 ± 0.3 | 5.3 ± 0.5 | 6.9 ± 0.8 | 8.4 ± 0.95 |
| Inhibitor | 1 | 1.64 ± 0.1 | 3.3 ± 0.25 | 3.9 ± 0.7 | 5.3 ± 0.7 | 7.7 ± 1.7 |
| Irradiation | 1 | 1.65 ± 0.1 | 1.9 ± 0.1 | 1.85 ± 0.1 | 2.35 ± 0.3 | 3.8 ± 0.4 |
| Inhibitor + irradiation | 1 | 1.4 ± 0.1 | 1.7 ± 0.2 | 1.84 ± 0.3 | 2.65 ± 0.6 | 4.6 ± 0.8 |

TABLE 7
Effects of 9-phenyl-symmetric-octahydroselenoxanthene and
localized irradiation on number of lung metastases.

| | | Average number of lung |
|---|---|---|
| Treatment | Number of mice | metastases |
| Control | 7 | 30.7 ± 4.6 |
| Inhibitor | 10 | 33.5 ± 2.5 |
| Irradiation | 9 | 11.7 ± 1.9 |
| Inhibitor + irradiation | 9 | 13.4 ± 1.8 |

Utilization of the invention—administration of 9-phenyl-symmetric-octahydroselenoxanthene—will increase the efficiency of radiation and cytostatic treatments by alleviating their toxic effects on hematopoiesis. Application of 9-phenyl-symmetric-octahydroselenoxanthene does not increase the tumor's resistance to radiation treatment.

While this invention has been particularly shown and described with references to preferred embodiments thereof, it will be understood by those skilled in the art that various changes in form and details may be made therein without departing from the scope of the invention encompassed by the appended claims.

What is claimed is:

1. Use of a selenium compound having a molecular structure: 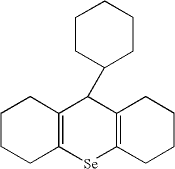
as an inhibitor of hematopoietic progenitor cell differentiation.

2. Use of the selenium compound according to claim 1, wherein the inhibitor is administered 4 to 9 days prior to radiation or chemotherapy.
